(19) United States
(12) Patent Application Publication
(10) Pub. No.: US 2004/0228444 A1
(43) Pub. Date: Nov. 18, 2004

(54) X-ray diagnostic apparatus with image computer for direction filtering

(76) Inventors: Stefan Bohm, Oberasbach (DE); Alois Nottling, Pottenstein (DE)

Correspondence Address: SCHIFF HARDIN, LLP, PATENT DEPARTMENT, 6600 SEARS TOWER, CHICAGO, IL, 60606-6473, US

(21) Appl. No.: 10/788,520

(22) Filed: Feb. 27, 2004

(30) Foreign Application Priority Data
Feb. 28, 2003 (DE) 103 09 166.1

(51) Int. Cl. H05G 1/64; A61B 6/00

(52) U.S. Cl. 378/98.2

(57) ABSTRACT

An x-ray diagnostic device has an x-ray source to generate x-rays, an x-ray detector to acquire x-ray images and transduce them into an electrical signal sequence, an image system to process the electrical signal sequence, and a playback device. The image system has a unit to detect edges in individual x-ray images and a unit to filter the individual x-ray images along these edges. The individual image processing can ensue in real time. Based on a signal adaptation by the detection of the weighting of the edges, the filtering can be implemented along these edges.

### BACKGROUND OF THE INVENTION

1. Field of the Invention

The present invention concerns an x-ray diagnostic apparatus of the type having an x-ray device to generate x-ray radiation, an x-ray detector to acquire x-ray images and transduce them into an electrical signal sequence, an image system to process the electrical signal sequence, and a playback device.

2. Description of the Prior Art

To acquire fluoroscopic x-ray images with navigation using a guidewire and a catheter, typically the least radiation dose is employed. Due to this low dose, a very low signal-to-noise ratio results, such that the image quality is very significantly limited.

Conventionally, such x-ray images undergo a time integration, for example by formation of a running average value image described, for example, in U.S. Pat. No. 5,495,514. A disadvantage of this technique, however, is that movement sharpening and ghost images ensue. As an alternative, spatial low-pass filtering is known, but this technique causes a blurring of the subjects (for example of the vessel edges) that has to be accepted as a compromise.

### SUMMARY OF THE INVENTION

An object of the present invention is to provide an image computer of an x-ray diagnostic device of the initially-described type that allows image series to be processed in real time with the signal-to-noise ratio being improved.

This object is inventively achieved in an x-ray diagnostic apparatus of the type initially described having an image system having a device that detects edges in individual x-ray images and a device to filter the individual x-ray images along these edges. Unlike conventional techniques, no ghost images result due to the individual image processing, even in image series. A signal adaptation ensues by the detection of the weighting of the edges. The filtering is implemented along these edges.

It has proven to be advantageous when the device for filtering implements an averaging over a number of pixels. The averaging can ensue in accordance with the invention by means of a directed mask.

The device for edge detection can be undertaken by a unit for variance measurement to which is attached a unit to determine the minimum of the variances, to determine the optimal direction.

It has proven to be advantageous when the device for edge detection includes a unit for interpolation of pixel values of a discrete pixel raster to generate an output signal with a sub-pixel resolution in the direction determination.

The determination of direction fields of the filter mask can ensue in accordance with the invention based on a reduced pixel count, whereby the direction fields can be interpolated for the low-pass filtering.

### DESCRIPTION OF THE DRAWINGS

FIG. 1 illustrates a known x-ray diagnostic device.

FIG. 2 is a block diagram of an image system according to the invention, suitable for use in the device of FIG. 1.

FIG. 3 shows pixel raster for explaining the invention.

FIGS. 4 through 11 illustrate respective filter masks to explain the invention.

### DESCRIPTION OF THE PREFERRED EMBODIMENTS

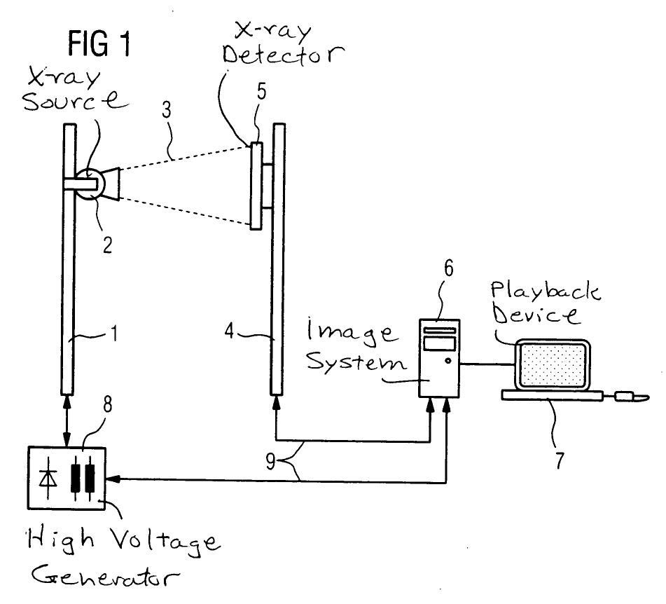

FIG. 1 illustrates an x-ray diagnostic device known from German OS 195 27 148, with a first stand 1 on which an x-ray radiator 2 is mounted so as to be adjustable in height, which generates a conical x-ray beam 3, and a second stand 4 to which an x-ray detector 5 is mounted, such that its height is aligned to the x-ray radiator 5, so that the x-ray beam 3 is incident on the x-ray detector 5. The output signal of the x-ray detector 5 is supplied to an image computer or image system 6. The image system 6 can include computers, transducers, image memories and processing circuits. It is connected with a control monitor 7 to reproduce the acquired x-ray images. A high-voltage generator 8 supplies the x-ray tube of the x-ray radiator 2 with high voltage and filament voltage. The image system 6 is connected with the remaining components of the x-ray diagnostic device via control and data lines 9.

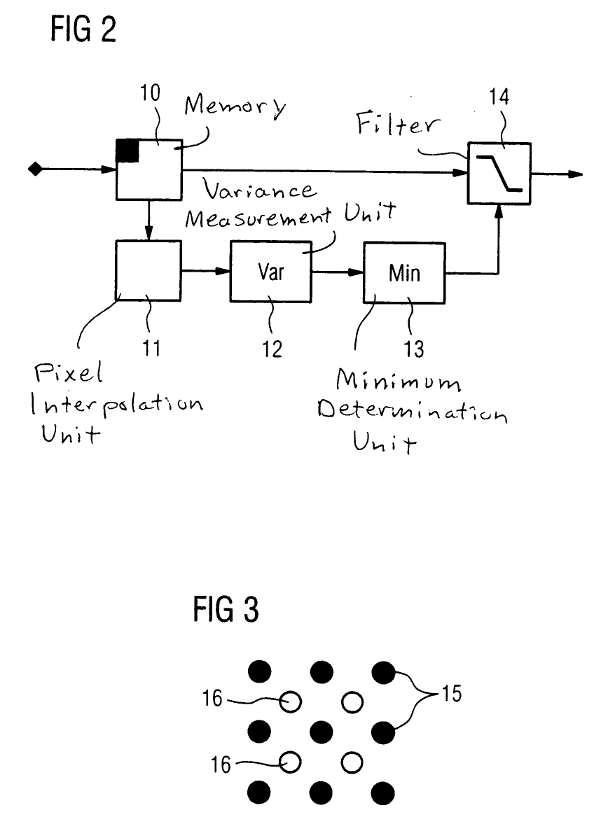

An image system according to the invention, suitable for use as the image system 6 of the x-ray diagnostic device according to FIG. 1, has an image memory 10 (shown in FIG. 2) to which the input signal is supplied. Connected to the image memory 10 is a unit 11 that interpolates pixel values and that is connected with a unit 12 for variance measurement. The output signal of the unit 12 for variance measurement is supplied to a unit 13 for determination of the minimum of the variances, the output of which controls a unit 14 for filtering.

The interpolation in the unit 11 thereby ensues such that intermediate values that form a sub-pixel raster 16 are calculated from a discrete pixel raster 15 shown in FIG. 3. The sub-pixel raster 16 is the region between the points of entries of existing discrete pixel raster 15.

For an example with eight pixels in the filter mask, the unit 12 for variance measurement calculates, at pixel values pᵢ, the average value \overline{P} of the pixel values, which is subtracted from the pixel value pᵢ; and the result is squared and from this the average value is formed. This procedure is represented by the equation:

$$Var = {\sum }_{i = 0}^{7} {( {p}_{i} - \overline{P} )}^{2} / 8$$

The variance measurement thereby ensues directionally dependent, i.e. within the filter masks.

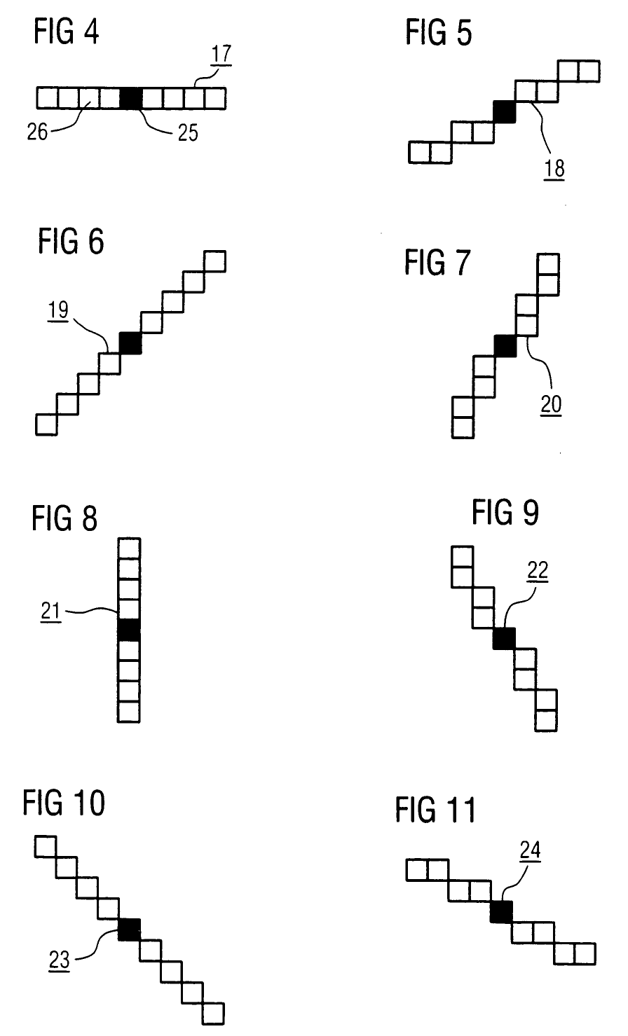

The minimum at these variances is determined by the unit 13, from which the direction of edges results. This result is supplied to the unit 14 for directed filtering, in which filtering ensues along the edges by averaging of a directed filter mask shown in FIGS. 4 through 11.

Examples of direction fields 17 through 24 of the filter masks are shown in FIG. 4 through 11 for eight different directions. They show that pixels 26 (that result from averaging of the new value for the current pixel 25) that are adjacent around each current pixel 25 (pᵢ) are acquired. Other and more different directions, as well as higher numbers of pixels 25 and 26 to be averaged, are also possible. Non-discrete filter masks also can be used for which interpolation is not necessary.

As a result the individual image processing, (for example) no ghost images result as in conventional techniques, even in image series. A signal adaptation ensues by the detection of the weighting of the edges. A filtering then ensues along these edges, for example an averaging over a number of pixels. Due to the real-time capability, the method is also suited for interventional operations. The quantities that are relevant for image quality, such as strength and characteristic, are adjustable on the user interface.

Given the edge detection by variance measurement and determination of the optimal direction by determination of the minimum of the variances, it is achieved, based on the direction determination, that in spite of the noise the filter masks 17 through 23 align along edges, for example of vessels. Interesting structures are thereby retained in spite of strong noise suppression. Further advantages result by the interpolation of missing pixel values given the direction determination on sub-pixel precision or by limitation to the discrete pixel raster 15 by means of optimized filter masks 17 through 23. The direction determination ensues based on a reduced number of pixel, whereby the direction field 17 through 23 is interpolated for the subsequent low-pass filtering. The quantities relevant for image qualities, such as strength (dissolving factor at the original) and characteristic (core size) are adjustable at the user interface.

This image processing ensues line-dependent and/or pixel-dependent, or with image delay, and can be combined with other pixel-dependent algorithms.

The described device can also be implemented as software in a digital signal processor (DSP) such that it enables the real-time image processing.

Although modifications and changes may be suggested by those skilled in the art, it is the intention of the inventors to embody within the patent warranted hereon all changes and modifications as reasonably and properly come within the scope of their contribution to the art.

We claim as our invention:

1. An x-ray diagnostic apparatus comprising:
an x-ray source that emits x-rays;
an x-ray detector disposed such that said x-rays are incident thereon after passing through a subject, said x-ray detector acquiring a plurality of x-ray images formed by the x-rays incident thereon and converting said plurality of x-ray images into an electrical signal sequence;
an imaging system supplied with said electrical signal sequence for processing said electrical signal sequence to form video signals, said imaging system comprising an edge detector for detecting edges in individual x-ray images in said plurality of x-ray images and a filter for filtering said individual x-ray images along the edges detected by said edge detector, to generate said video signals; and
a playback device supplied with said video signals.

2. An x-ray diagnostic apparatus as claimed in claim 1 wherein each of said individual x-ray images is composed of a plurality of pixels, each having a pixel value, and wherein said filter forms an average of a number of said pixel values, less than said plurality of pixels.

3. An x-ray diagnostic apparatus as claimed in claim 2 wherein said filter forms said average using a directed mask that causes said averaging to ensue along a direction set by said directed mask.

4. An x-ray diagnostic apparatus as claimed in claim 3 wherein said edge detector comprises a variance measurement unit for making a variance measurement of said pixel values in each of said x-ray images, and a unit for determining a minimum of said variance measurement, said minimum determining said direction to be set by said directed mask.

5. An x-ray diagnostic apparatus as claimed in claim 4 wherein said edge detector further comprises an interpolator for interpolating pixel values of a pixel raster in each of said x-ray images to generate a sub-pixel raster in said direction.

6. An x-ray diagnostic apparatus as claimed in claim 5 wherein said variance measurement unit forms said variance over a selected number of pixels, less than said plurality of pixels.

7. An x-ray diagnostic apparatus as claimed in claim 6 wherein said filter is a low-pass filter and wherein said direction is determined by high interpolation of said pixel values.
